(19) United States
(12) Patent Application Publication
(10) Pub. No.: US 2014/0273104 A1
(43) Pub. Date: Sep. 18, 2014

(54) PRETREATMENT AND FRACTIONATION OF LIGNOCELLULOSIC BIOMASS

(71) Applicants: Praveen PARIPATI, Reston, VA (US); Anantharam Prasad DADI, Toledo, OH (US)

(72) Inventors: Praveen PARIPATI, Reston, VA (US); Anantharam Prasad DADI, Toledo, OH (US)

(73) Assignee: SuGanit Systems, Inc., Reston, VA (US)

(21) Appl. No.: 13/841,707

(22) Filed: Mar. 15, 2013

(51) Int. Cl. C12P 19/14; C07G 1/00

(52) U.S. Cl. CPC C12P 19/14; C07G 1/00
USPC 435/99; 162/14

(57) ABSTRACT

Method and apparatus for enhanced production of sugars and lignin via fractionation of lignocellulosic biomass through sequential ionic liquid pretreatment and mild alkaline treatment. The resulting biomass is easily fractionated and amenable to efficient and rapid hydrolysis and catalytic conversion to valuable products with high recovery of the enzymes used in the hydrolysis.

### FIELD OF THE INVENTION

The present invention relates the sequential treatment of lignocellulosic biomass with ionic liquid pretreatment followed by mild alkaline treatment for efficient generation of cellulosic material and lignin fractions. The resulting cellulosic material may be efficiently and rapidly catalytically converted to sugars, fuels, and chemicals and the lignin residue may be converted to chemicals and fuels.

### BACKGROUND OF THE INVENTION

Lignocellulose is the major structural component of plants and comprises cellulose, hemicellulose, and lignin. In lignocellulosic biomass, crystalline cellulose fibrils are embedded in a less well-organized hemicellulose matrix which, in turn, is surrounded by an outer lignin seal. Lignocellulosic biomass is an attractive feed-stock because it is an abundant, domestic, renewable source that can be converted to liquid transportation fuels, chemicals and polymers. The major constituents of lignocellulose are: (1) hemicellulose (20-30%), an amorphous polymer of five and six carbon sugars; (2) lignin (5-30%), a highly cross-linked polymer of phenolic compounds; and (3) cellulose (30-40%), a highly crystalline polymer of cellobiose (a glucose dimer). Cellulose and hemicellulose, when hydrolyzed into their monomeric sugars, can be converted into ethanol fuel through well-established fermentation technologies. These sugars also form the feedstocks for production of a variety of chemicals and polymers. The lignin may also be recovered for use in the production of feedstock or used a fuel. The complex structure of biomass requires proper treatment to enable efficient hydrolysis (e.g., saccharification) of cellulose and hemicellulose components into their constituent sugars.

Practical means of producing chemicals and fuels from lignocellulosic biomass are limited due to the recalcitrant nature of cellulose in lignocellulosic biomass. Crystalline cellulose and hemicellulose are tightly sealed in the highly crosslinked lignin polymer, which acts as a physical barrier towards any chemical or biological attack on the carbohydrates in the biomass. Lignin is linked to carbohydrates via covalent and hydrogen bonds making biomass degradation difficult. Current treatment approaches suffer from slow reaction rates of cellulose hydrolysis (e.g., using the enzyme cellulase) and low sugar yields. Wyman, et al. (2005) Bioresource Technology 96: 1959-1966).

Contacting lignocellulosic biomass with hydrolyzing enzymes generally results in cellulose hydrolysis yields that are less than 20% of possible results. Hence, some “pretreatment” of the biomass may be carried out prior to attempting the enzymatic hydrolysis of the cellulose and hemicellulose in the biomass. Pretreatment refers to a process that converts lignocellulosic biomass from its native form, in which it is recalcitrant to cellulase enzyme systems, into a form for which cellulose hydrolysis is effective. Compared to untreated biomass, effectively pretreated lignocellulosic materials are characterized by an increased surface area (porosity) accessible to cellulase enzymes, and solubilization or redistribution of lignin. Increased porosity results mainly from a combination of disruption of cellulose crystallinity, hemicellulose disruption/solubilization, and lignin redistribution and/or solubilization. The relative effectiveness in accomplishing at least some of these factors differs greatly among different existing pretreatment processes.

The purpose of the pretreatment is to significantly disrupt the structure of biomass in order to: (a) reduce the crystallinity of cellulose, (b) increase accessibility/susceptibility of cellulose and hemicellulose chains to enzymes/catalysts by increasing the surface area/porosity and (c) remove lignin. U.S. Pat. No. 8,030,030. Several thermo-chemical biomass pretreatments techniques were investigated over the past few decades for improving the digestibility of this highly recalcitrant biomass. These include dilute acid, steam explosion, hydrothermal processes, “organosolv” processes involving organic solvents in an aqueous medium, ammonia fiber explosion (AFEX), strong alkali processes using a base (e.g., ammonia, NaOH or lime), and highly-concentrated phosphoric acid treatment. Many of these methods do not disrupt cellulose crystallinity, an attribute vital to achieving rapid cellulose digestibility. Also, some of these methods are not amenable for efficient recovery of the chemicals employed in the pretreatment.

Despite of many improvements in biomass pretreatment technologies, rapid production of monomeric sugars in high yields is still an area of active research. Majority of the pretreatments at cost effective strategies do not disrupt cellulose crystallinity, an attribute vital to achieving rapid hydrolysis rates. Therefore, enzymatic conversion of pretreated biomass from majority of these technologies still requires 48 to 168 hours to produce high yields. Other concentrated acid processes can enhance the hydrolysis rates, however, they are not practical to implement due to toxicity and recoverability issues. Therefore there exists a need in the art for a more efficient method of sugar and lignin generation from lignocellulosic biomass.

### SUMMARY OF THE INVENTION

In one embodiment, the method for the treatment of lignocellulosic biomass may comprise (a) mixing lignocellulosic biomass with an ionic liquid for a sufficient time and temperature to swell the lignocellulosic biomass without dissolving the lignocellulosic biomass in the ionic liquid; and (b) treating the swelled lignocellulosic biomass under mild alkaline treatment to separate the lignin from the cellulose and hemicellulose. In another embodiment, the lignocellulosic biomass may be washed after ionic liquid pretreatment in step (a) and before mild alkaline treatment in step (b).

In one embodiment, a method for conversion of the carbohydrates of lignocellulosic biomass to sugars may comprise (a) mixing lignocellulosic biomass in an ionic liquid (IL) swell but not dissolve the biomass; (b) applying radio frequency (RF) heating to the biomass to heat to a target temperature range; (c) applying ultrasonics, electromagnetic (EM), convective, conductive heating, or combinations thereof, to the lignocellulosic biomass to maintain the biomass at said target temperature range; (d) washing the treated lignocellulosic biomass; (e) subjecting said lignocellulosic biomass to mild alkaline treatment to release lignin from the cellulosic components; (f) washing the treated lignocellulosic biomass; and (g) hydrolyzing the treated lignocellulosic biomass to yield sugars.

In one embodiment, a method for the conversion of cellulose to sugar may comprise (a) mixing lignocellulosic biomass in an ionic liquid (IL) to swell the lignocellulosic biomass; (b) applying radio frequency (RF) heating to the lignocellulosic biomass to heat to a target temperature range; (c) applying ultrasonics, electromagnetic (EM), convective, conductive heating, or combinations thereof, to the lignocellulosic biomass to maintain the biomass at said target temperature range; (d) washing the pretreated lignocellulosic biomass; (e) subjected said lignocellulosic biomass to mild alkaline treatment to release lignin from the cellulosic components; (f) washing the treated lignocellulosic biomass; and (g) hydrolyzing the treated biomass to yield sugars and release lignin.

In one embodiment, a method for treatment of lignocellulosic biomass may comprise (a) incubating a biomass in a sufficient amount of an ionic liquid (IL) for a sufficient time and temperature to swell the biomass without dissolution of the biomass in the IL; (b) applying radio frequency (RF) heating to the lignocellulosic biomass to heat to a target temperature range; (c) applying ultrasonic heating to the lignocellulosic biomass to maintain the biomass at said target temperature range; (D) washing the pretreated lignocellulosic biomass; (e) subjecting said lignocellulosic biomass to mild alkaline treatment to release lignin from the cellulosic components; (f) washing the treated lignocellulosic biomass with a liquid non-solvent for cellulose that is miscible with water and the IL; and (g) contacting said washed treated lignocellulosic biomass with an aqueous buffer may comprise enzymes capable of hydrolyzing cellulose and hemicellulose to produce sugars.

In another embodiment, the mild alkaline treatment may comprise the addition of an alkaline agent. In another embodiment, the alkaline agent may be NaOH, aqueous ammonia, LiOH, Mg(OH)₂, Al(OH)₃, Ca(OH)₂, H₂O₂, NaS, Na₂CO₃, or a combination thereof. In another embodiment, the alkaline agent may be added at a concentration of 1, 2, 3, 4, 5, 6, 7, 8, 9, 10, 11, 12, 13, 14, 15, 16, 17, 18, 19, 20, or 10-20% by weight.

In another embodiment, the mild alkaline condition comprise a pH of about 8-11, pH 8-10, pH 9-11, pH 9-10, pH 10-11, pH 9.5-10.5, 8, 8.25, 8.5, 8.75, 9, 9.25, 9.5, 9.75, 10, 10.25, 10.5, 10.75, or 11. In another embodiment, the pH may be about 8, 8.25, 8.5, 8.75, 9, 9.25, 9.5, 9.75, 10, 10.25, 10.5, 10.75, 11, 11.5, 12, 12.5 or 13.

In another embodiment, the alkaline treatment may be at a temperature of at least about 10° C., 20° C., 30° C., 40° C., 50° C., 60° C., 70° C., 40° C.-60° C., 50° C.-70° C., 50° C.-60° C., 40° C.-70° C., 40° C., 50° C., 70° C., 40° C.-60° C., 50° C.-70° C., 50° C.-60° C., or 40° C.-70° C.

In another embodiment, the mild alkaline treatment may be for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 1-60, 1-30, 1-20, 5-10, 1-10, 10, 20, 30, 40, 50, or 60 minutes. In a further embodiment, the mild alkaline treatment may comprise about 0.5%, 1%, 2%, 3%, 4%, 5% NaOH or KOH at about a pH of 9-10 for about 30 minutes at 40-50° C.

In one embodiment, the additional heating may comprise intermittent agitation during heating.

In one embodiment, the ionic liquid may be molten at a temperature ranging from about 10° C. to 160° C. and may comprise cations or anions. In another embodiment, the ionic liquid may comprise a cation structure that includes ammonium, sulfonium, phosphonium, lithium, imidazolium, pyridinium, picolinium, pyrrolidinium, thiazolium, triazolium, oxazolium, or combinations thereof. In another embodiment, the ionic liquid may comprise a cation selected from imidazolium, pyrrolidinium, pyridinium, phosphonium, ammonium, or a combination thereof. In another embodiment, the ionic liquid (IL) may be 1-n-butyl-3-methylimidazolium chloride, 1-allyl-3-methyl imidazolium chloride, 3-methyl-N-butylpyridinium chloride, 1-ethyl-3-methyl imidazolium acetate, 1-ethyl-3-methyl imidazolium propionatem, or combinations thereof.

In one embodiment, the method further may comprise treating said treated biomass with a biochemical reagent to convert the cellulose and hemicellulose to sugars. In another embodiment, the sugars may be hexose and/or pentose sugars.

In another embodiment, the biochemical reagent may be an enzyme. In another embodiment, the biochemical reagent may be an enzyme mixture of hemicellulases, cellulases, endo-glucanases, exo-glucanases, and 1-β-glucosidases. In another embodiment, the cellulase may be cellobiohydrolase, endocellulase, exocellulase, cellobiase, endo-beta-1,4-glucanase, beta-1,4-glucanase, or mixtures thereof. In another embodiment, the hemicellulase may be laminarinase, lichenase, xylanase, or mixtures thereof. In another embodiment, the enzyme mixture further may comprise xylanases, arabinases, or mixtures thereof.

In one embodiment, the biochemical reagent may be a thermophilic enzyme. In another embodiment, the thermophilic enzyme may be active up to about 70° C. In another embodiment, the enzyme may be added at a concentration of about 0.5, 1, 2, 3, 4, or 5% by weight.

In one embodiment, the enzyme may be recovered. In another embodiment, at least 90, 91, 92, 93, 94, 95, 96, 97, 98, or 99% of the enzyme may be recovered.

In one embodiment, the enzyme may be reused. In a further embodiment, the enzyme may be reused for about 16-20 hydrolysis cycles. In a further embodiment, the sugars may be converted to renewable fuels, chemicals and materials.

In a further embodiment, the heating may comprise at least two phases, a first phase may comprise application of electromagnetic (EM) heating, optionally a variable frequency in the electromagnetic spectrum, variable frequency heating, radiofrequency (RF) heating, or a combination thereof, and a second phase may comprise application of ultrasonics, electromagnetic (EM), convective, conductive heating, or combinations thereof.

In a further embodiment, the application of radiofrequency heating may be for about at least 5-10 seconds, 1-30 minutes, 5-30 minutes, or 20-240 minutes.

In a further embodiment, the application of ultrasonics, electromagnetic (EM), convective, conductive heating, or combinations thereof, may be for about at least 3-30 minutes, 5-30 minutes, or 3-4 hours.

In another embodiment, the method may further comprise washing the treated biomass. In yet another embodiment, the washing may comprise washing the biomass with a liquid non-solvent for cellulose that is miscible with water and the ionic liquid (IL). In another embodiment, the liquid non-solvent used for washing may be water, an alcohol, acetonitrile or a solvent which dissolves the IL. In yet another embodiment, the wash may be recovered and treated with RF heating to dehydrate the ionic liquid.

In yet another embodiment, the ionic liquid (IL) may be 1-n-butyl-3-methylimidazolium chloride, 1-allyl-3-methyl imidazolium chloride, 3-methyl-N-butylpyridinium chloride, 1-ethyl-3-methyl imidazolium acetate, 1-ethyl-3-methyl imidazolium propionatem, or combinations thereof.

In one embodiment, the biomass may be agricultural residues including but not limited to corn stover, wheat straw, bagasse, rice hulls, or rice straw; wood and forest residues including but not limited to pine, poplar, douglas fir, oak, saw dust, paper/pulp waste, or wood fiber; kudzu; herbaceous energy crops including but not limited to switchgrass, reed canary grass, or miscanthus; lingocellulosic biomass including but not limited to may comprise lignin, cellulose, and hemicellulose; plant biomass, or mixtures thereof.

In another embodiment, the heating may comprise at least two phases, a first phase may comprise application of radiofrequency (RF) heating and a second phase may comprise application of ultrasonics, electromagnetic (EM), convective, conductive heating, or combinations thereof. In another embodiment, the application of radiofrequency heating may be for about at least 5-10 seconds, 1-30 minutes, 5-30 minutes, or 20-240 minutes. In another embodiment, the application of ultrasonics, electromagnetic (EM), convective, conductive heating, or combinations thereof may be for about at least 3-30 minutes, 5-30 minutes, or 3-4 hours. In another embodiment, the electromagnetic energy may be applied at a power of 100-1000 W, 1 KW-10 KW, or 5 KW-1 MW. In another embodiment, the radiofrequency may comprise a frequency between about 1-900 MHz, 300 kHz-3 MHz, 3-30 MHz, 30-300 MHz, 13, 13.56, 27, 27.12, 40, or 40.68 MHz. In another embodiment, the radiofrequency may penetrate the biomass to about 0.001 to 2.0 meters thickness. In another embodiment, the biomass may be heated to a temperature of at least about 1-300° C., 50° C.-100° C., 60° C.-130° C., 80° C.-175° C., or 100° C.-240° C. In another embodiment, the biomass may be treated with radiofrequency for at least about 1 minute to 100 hours, 1-60 minutes, 1-24 hours, 5-10 minutes, 5-30 minutes, 10-50 minutes, 5 minutes to 3 hours, 1-3 hours, 2-4 hours, 3-6 hours, or 4-8 hours.

In one embodiment, the method may further comprise washing the treated biomass. In another embodiment, the washing may comprise washing the biomass with a liquid non-solvent for cellulose that is miscible with water and the ionic liquid (IL). In another embodiment, the liquid non-solvent used for washing may be water, an alcohol, acetonitrile or a solvent which dissolves the IL and thereby may extract the IL from the biomass. In another embodiment, the alcohol may be ethanol, methanol, butanol, propanol, or mixtures thereof.

In one embodiment, the ionic liquid may be recovered from the liquid non-solvent by a method selected from one or more of activated charcoal treatment, distillation, membrane separation, electro-chemical separation techniques, sold-phase extraction liquid-liquid extraction, or a combination thereof. In another embodiment, the ionic liquid may be recovered from the liquid non-solvent by application of electromagnetic heating including but not limited to radiofrequency heating, that dehydrates the ionic liquid. In another embodiment, the method may comprise the further step of reusing the recovered IL for treating more biomass including but not limited to wherein at least 90, 91, 92, 93, 94, 95, 96, 97, 98, or 99% of the IL may be recovered. In another embodiment, the ionic liquid may have a water content not exceeding about 1, 2, 3, 4, 5, 6, 7, 8, 9, 10, 11, 12, 13, 14, 15, 16, 17, 18, 19, 20, 21, 22, 23, 24, or 25%.

In one embodiment, the method may comprise incubating the biomass in a sufficient amount of an ionic liquid (IL) for a sufficient time to swell the biomass. In one embodiment, the method may comprise incubating the biomass in a sufficient amount of an ionic liquid (IL) for a sufficient time to swell the biomass but not dissolve the biomass. In one embodiment, the biomass may be not dissolved in the ionic liquid.

In one embodiment, the biomass may be heated by heating with agitation, ultrasonics heating, electromagnetic (EM) heating, convective heating, conductive heating, microwave irradiation, or a combination thereof including but not limited to with intermittent agitation during heating.

In one embodiment, the ionic liquid may be molten at a temperature ranging from about 10° C. to 160° C. and may comprise cations or anions. In another embodiment, the ionic liquid may comprise a cation structure that includes ammonium, sulfonium, phosphonium, lithium, imidazolium, pyridinium, picolinium, pyrrolidinium, thiazolium, triazolium, oxazolium, or combinations thereof. In another embodiment, the ionic liquid may comprise a cation selected from imidazolium, pyrrolidinium, pyridinium, phosphonium, ammonium, or a combination thereof. In another embodiment, the ionic liquid (IL) may be 1-n-butyl-3-methylimidazolium chloride, 1-allyl-3-methyl imidazolium chloride, 3-methyl-N-butylpyridinium chloride, 1-ethyl-3-methyl imidazolium acetate, 1-ethyl-3-methyl imidazolium propionatem, or combinations thereof.

In one embodiment, the method may be a continuous process. In another embodiment, the method may be a batch process.

In one embodiment, the conditions of said biomass undergoing radiofrequency (RF) heating may be monitored by means of sensors including but not limited to a liquid flow rate sensor, thermocouple sensor, temperature sensor, salinity sensor, or combinations thereof. In another embodiment, the method may comprise adjusting the amount of ionic liquid, the time of incubation, or the temperature of the biomass.

In one embodiment, the method may further comprise treating said treated biomass with biochemical reagents including but not limited to an enzyme, to convert the cellulose and hemicellulose to sugars including but not limited to hexose and pentose sugars. In another embodiment, the biochemical reagent used to convert the cellulose and hemicellulose may be an enzyme including but not limited to an enzyme mixture of hemicellulases, cellulases, endo-glucanases, exo-glucanases, and 1-β-glucosidases. In another embodiment, the cellulase may be cellobiohydrolase, endocellulase, exocellulase, cellobiase, endo-beta-1,4-glucanase, beta-1,4-glucanase, or mixtures thereof.

In another embodiment, the hemicellulase may be laminarinase, lichenase, xylanase, or mixtures thereof. In another embodiment, the enzyme mixture may further comprise xylanases, arabinases, or mixtures thereof. In another embodiment, the biochemical reagents are thermophilic enzymes including but not limited to enzymes that are active up to about 70° C. In another embodiment, the biomass may be heated to at least about 50-100° C., 40° C., 55° C., or 70° C.

In another embodiment, the sugars may be converted to renewable fuels, chemicals and materials including but not limited to ethanol, butanol, lactic acid, gasoline, biodiesel, methane, hydrogen, electricity, plastics, composites, protein, drugs, fertilizers or other components thereof.

In another embodiment, the chemicals may be succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof.

In one embodiment, the method may further comprise recovering the enzymes. In another embodiment, at least about 60, 70, 80, 90, 91, 92, 92, 93, 94, 95, 96, 97, 98, 99, or 100% of the enzymes may be recovered.

In one embodiment, the treatment produces a solid residue may comprise proteins, lignin, and/or ash. In another embodiment, the proteins and/or lignin may be recovered for use as feedstock or fuel.

In one embodiment, the method further may comprise treating said treated biomass with chemical reagents to convert the cellulose and hemicellulose to sugars including but not limited to hexose and pentose sugars. In another embodiment, the sugars may be converted to chemicals including but not limited to succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof.

In one embodiment, the reactor may be loaded with a high level of biomass. In another embodiment, the biomass may comprise high solids loadings at least about 10%, 20%, 30%, 40%, 50%, 60%, 70%, 80%, 90%, or 95% w/w. In a further embodiment, the biomass may be loaded at high solids loading at approximately 30% w/w.

In one embodiment, the biomass may be comminuted to smaller sized particles. In another embodiment, the biomass may be comminuted to smaller sized particles prior to mixing with an ionic liquid. In a further embodiment, the biomass may be comminuted to small particles about 0.1-20 mm, 0.1-2 mm, or about 5 mm in size.

In another embodiment, the treated biomass may be further processed to yield renewable fuels, chemicals and materials including but not limited to ethanol, butanol, lactic acid, gasoline, biodiesel, methane, hydrogen, electricity, plastics, composites, protein, drugs, fertilizers or other components thereof.

### BRIEF DESCRIPTION OF THE DRAWINGS

FIG. 1 depicts a schematic of the pretreatment on lignocellulosic biomass. (A) the general structure of lignoscellulosic biomass, (B) pretreatment depicting partial breakdown, and (C) improved pretreatment depicting complete disruption of the biomass structure including the elimination of cellulose crystallinity.

FIG. 2A depicts an exemplary method for processing biomass comprising mixing with ionic liquid to swell but not dissolve the biomass in the IL followed by mild alkaline treatment at about pH 8-11, optionally heating by radio frequency, ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof, optionally about 5-30 minutes, optionally repeating heating, washing the biomass, optionally recovering the IL. The mild alkaline treatment produces two outputs, a hydrolystate stream comprising cellulose and hemicellulose and a residue comprising lignin. The hydrolystate stream comprising cellulose and hemicellulose may be directed to further processing by the addition of cellulase and hemicellulases to their constituent monomeric sugars (e.g., five and six carbon sugars), optionally recovery of the added enzymes, to produce chemicals or biofuels. The residual solids (e.g., proteins and/or lignin) may be directed to further processing to produce feedstock or biofuels. The ionic liquid and enzymes may be reclaimed and reused.

FIG. 2B depicts an exemplary method for processing biomass comprising mixing with ionic liquid to swell but not dissolve the biomass in the IL followed by mild alkaline treatment at about pH 8-11, optionally heating by radio frequency irradiation to reach a target temperature range, optionally repeated, maintaining the temperature of the biomass using of ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof, optionally about 5-30 minutes, optionally repeated, washing the biomass, optionally recovering the IL and dehydrating the IL by application of radiofrequency heating. The mild alkaline treatment produces two outputs, a hydrolystate stream comprising cellulose and hemicellulose and a residue comprising lignin. The hydrolystate stream comprising cellulose and hemicellulose may be directed to further processing by the addition of cellulase and hemicellulases to their constituent monomeric sugars (e.g., five and six carbon sugars), optionally recovery of the added enzymes, to produce chemicals or biofuels. The residual solids (e.g., proteins and/or lignin) may be directed to further processing to produce feedstock or biofuels. The ionic liquid and enzymes may be reclaimed and reused.

FIG. 3 depicts exemplary cation and anion components of ionic liquids.

FIG. 4 depicts the percentage of glucan and xylan conversion to monomeric sugars. (A) pretreated (PT) wheatstraw plus alkaline treatment (CT) at 75° C. for 60 minutes; (C) pretreated (PT) wheatstraw with no alkaline treatment (CT); (C) no pretreatment (UT, untreated) wheatstraw plus alkaline treatment at 75° C. for 60 minutes, and (D) untreated wheatstraw.

### DETAILED DESCRIPTION OF THE PREFERRED EMBODIMENTS

In order that the invention herein described may be fully understood, the following detailed description is set forth. Various embodiments of the invention are described in detail and may be further illustrated by the provided examples. Additional viable variations of the embodiments can easily be envisioned.

### DEFINITIONS

Unless defined otherwise, all technical and scientific terms used herein have the same meaning as those commonly understood by one of ordinary skill in the art to which this invention belongs.

As used in the description herein and throughout the claims that follow, the meaning of “a,” “an,” and “the” includes plural reference unless the context clearly dictates otherwise.

“Biomass,” as used herein, refers broadly to any biological material. Biomass encompasses substrates containing organic components which can be used in production of renewable fuels, chemicals and materials such as ethanol, butanol, lactic acid, gasoline, biodiesel, methane, hydrogen, plastics, composites, protein, drugs, fertilizers or other components thereof. Biomass may be agricultural residues including but not limited to corn stover, wheat straw, bagasse, rice hulls, or rice straw; wood and forest residues including but not limited to pine, poplar, douglas fir, oak, saw dust, paper/pulp waste, or wood fiber; feedstock (e.g., woody biomass and agricultural biomass); kudzu; algae including but not limited to red algae; herbaceous energy crops including but not limited to switchgrass, reed canary grass, or miscanthus; lingocellulosic biomass; plant biomass; or mixtures thereof. Biomass may be lignocellulosic biomass comprising cellulose, hemicellulose, and lignin.

“Ionic liquids” as used herein, refers broadly to room temperature liquids that contain only ions and are molten salts stable up to 300° C. Sheldon (2001) Chem. Commun. 23: 2399-2407.

“Lignocellulosic biomass” as used herein, refers broadly to plant biomass that is composed of cellulose, hemicellulose, and lignin. The carbohydrate polymers (e.g., cellulose and hemicelluloses) are tightly bound to the lignin. Lignocellulosic biomass can be grouped into four main categories: agricultural residues (e.g., corn stover and sugarcane bagasse), dedicated energy crops, wood residues (e.g., sawmill and paper mill discards), and municipal paper waste.

“Mild alkaline treatment,” as used herein, refers broadly to a process of treating biomass with an alkaline agent (e.g., Ca(OH)₂, Mg(OH)₂, Al(OH)₃, NaOH) at low concentration (e.g., 1-5%) for a brief time (e.g., about 30 minutes) at a lower temperature (e.g., 40-70° C.) at about pH 8-13. This is in contrast to prior methods where the agents were used at high concentration, high temperature, for longer periods of time (e.g., 30% at 120-150° C. for 3-4 weeks at pH>11).

“Pretreatment of biomass,” as used herein, refers broadly to a process of changing the physiochemical structure of biomass to make it amenable for efficient conversion to their monomeric valuable products.

“Radiofrequency (RF) heating,” as used herein, refers broadly to application of electromagnetic field to biomass/products/dielectric materials at frequencies from about 1-300 MHz.

“Electromagnetic energy (EM),” as used herein, refers broadly to a form of energy that is reflected or emitted from objects in the form of electrical and magnetic waves that can travel through space. There are many forms of electromagnetic energy including gamma rays, x rays, ultraviolet radiation, visible light, infrared radiation, microwaves, and radio waves (radiofrequency).

“Ultrasonics” or “ultrasonic waves,” as used herein, refers broadly to sound waves (mechanical waves) with high frequency about between 15 kHz to 40 kHz (e.g., about 20 kHz) and low amplitude about between 0.0001-0.025 mm.

### Biomass Treatment Combination of IL Pretreatment and Mild Alkaline Treatment

The present invention provides a method for the treatment of biomass to yield useful chemicals comprising the combination of ionic liquid pretreatment followed by mild alkaline treatment.

This invention provides an efficient biomass disruption/fractionation strategy employing sequential ionic liquid pretreatment followed by mild alkaline treatment process which (a) can be used for treating any lignocellulosic biomass substrates, (b) results in efficient cellulosic material and lignin fraction generation at mild conditions (of temperature, pressure, time, chemical, solvents) enabling catalytic conversions of all constituents of biomass in a single or sequential steps (c) results in a de-crystallized and swollen cellulose for catalytic conversion to monomeric sugars or chemicals, (d) results in enhanced production of monomeric sugars, (e) results in a catalytically convertible lignin fraction for generation of aromatic chemicals, (f) results in purer fractions of cellulosic pulp and lignin fractions, (g) results in lower reagent, chemical and catalyst requirements, (h) enables economic recovery of catalysts and chemicals. The inventors surprisingly discovered that the combination of ionic liquid pretreatment with mild alkaline treatment allows for improved treatment of biomass and unexpectantly resulted in higher enzyme recovery levels (e.g., >90%) and greater enzyme longevity (e.g., 16-20 uses versus 1 use).

### Ionic Liquid (IL)

Ionic liquids are liquids at room temperature and may contain only ions and are molten salts stable up to 300° C. See Sheldon (2001) Chem. Commun. 23: 2399-2407. They contain cations which are usually organic compounds and anions of inorganic or organic components such that the resulting salts are asymmetric. Because of poor packing associated with the asymmetric nature of ILs, crystal formation is inhibited and ILs remain liquids over a wide range of temperatures. A wide range of anions and cations can be employed to generate ILs with varied melting points, viscosities, thermal stabilities and polarities. Examples of some of the cations currently used include ammonium, sulfonium, phosphonium, lithium, imidazolium, pyridinium, picolinium, pyrrolidinium, thiazolium, triazolium oxazolium, or combinations thereof. Murugesan & Linhardt (2005) Current Organic Synthesis 2: 437-451. Ionic liquids are also liquid at <100° C., broad liquid range, almost no vapor pressure, high polarity, high dissolving power for organic and inorganic materials, good thermal, mechanical, and electrochemical stability, high heat capacity, non-flammable, and electrical conductivity.

Ionic liquids have extremely low volatility and when used as solvents, they do not contribute to emission of volatile components. Ionic liquids are considered environmentally benign solvents. ILs have been designed to dissolve cellulose and lignocellulose.

The ionic liquid treatment differs from the classic approach to the use of ionic liquids in that the aim is not to dissolve lignocellulose, but rather to contact it with the IL for times sufficient to mainly disrupt lignin sheathing and swell the remaining biomass structure significantly (at least 30%) but not dissolve the lignocellulose. This combination treatment enables the subsequent enzymatic hydrolysis process to proceed in a relatively short period of time as well as give quantitative yields of glucose and high yields of pentose sugars.

Any ionic liquid capable of disrupting the hydrogen bonding structure to reduce the crystallinity of cellulose in the biomass can be used in the treatment methods described herein may comprise a cation structure that includes imidazolium, pyrroldinium, pyridinium, phosphonium, ammonium, or a combination thereof and all functionalized analogs thereof. For example, the structure of triazolium as shown in FIG. 3 wherein each of R1, R2, R3, R4, and R5 may be hydrogen, an alkyl group having 1 to 15 carbon atoms or an alkene group having 2 to 10 carbon atoms, wherein the alkyl group may be substituted with sulfone, sulfoxide, thioether, ether, amide, hydroxyl, or amine and wherein A may be a halide, hydroxide, formate, acetate, propionate, butyrate, any functionalized mono- or di-carboxylic acid having up to a total of 10 carbon atoms, succinate, lactate, aspartate, oxalate, trichloroacetate, trifluoroacetate, dicyanamide, or carboxylate. Another example of the structure of IL is shown in FIG. 3 pyridine wherein each of R1, R2, R3, R4, R5, and R6 may be hydrogen, an alkyl group having 1 to 15 carbon atoms or an alkene group having 2 to 10 carbon atoms, wherein the alkyl group may be substituted with sulfone, sulfoxide, thioether, ether, amide, hydroxyl, or amine and wherein A may be a halide, hydroxide, formate, acetate, propanoate, butyrate, any functionalized mono- or di-carboxylic acid having up to a total of 10 carbon atoms, succinate, lactate, aspartate, oxalate, trichloroacetate, trifluoroacetate, dicyanamide, or carboxylate. The halide can be a chloride, fluoride, bromide or iodide.

Also an ionic liquid mixture with a composition described by Equation 1 may be used in the methods and systems described herein.

$${\sum }_{n = 1}^{20} {{[ {C}^{+} ]}_{n} [ {A}^{-} ]}_{n}$$

C⁺ denotes the cation of the IL and A⁻ denotes the anionic component of the IL In Equation 1. Each additional IL added to the mixture may have either the same cation as a previous component or the same anion as a previous component, of differ from the first only in the unique combination of the cation and anion. For example, consider below the five component mixture of ILs in which common cations and anions are used, but each individual IL component is different:

[BMIM⁺][Cl⁻]+[BMIM⁺][PF₆₋]+[EMIM⁺][Cl⁻]+[EM−IM⁺][PF₆₋]+[EMIM⁺][BF₄⁻]

The final mixture of ionic liquids will vary in the absolute composition as can be defined by the mole percent of various functionalized cations and anions. Therefore, the mixture may be comprised of varying weight percentages of each utilized component, as defined by Equation 1. The use of several such representative solvents for treating biomass may be 1-Ethyl-3-Methylimidazolium Propionate (EMIM-Pr) as described in U.S. Pat. No. 8,030,030. Also the ionic liquid 1-(4-sulfonic acid) butyl-3-methylimidazolium hydrogen sulfate may be used.

The ionic liquid may have a water content not exceeding about 1, 2, 3, 4, 5, 6, 7, 8, 9, 10, 11, 12, 13, 14, 15, 16, 17, 18, 19, 20, 21, 22, 23, 24, or 25%. Also, the ionic liquid may be recovered and reused.

Pretreatment of Lignocellulosic Biomass with Ionic Liquids

The pretreatment of lignocellulosic biomass by using Ionic Liquids (ILs) facilitates efficient and rapid enzymatic hydrolysis of its carbohydrates. The goal of the IL pretreatment process is not achieving any dissolution of lignocellulose, but contacting it with the IL for sufficient time to redistribute lignin and swell the remaining biomass structure to enhance the hydrolysis rate and conversion of cellulose and hemicellulose to their constituent sugars. Following saccharification with an appropriate enzyme mix, capable of converting all the carbohydrates in the pre-treated biomass to sugars, most of the solids left behind in the saccharification reactor represent the lignin portion of the biomass. This provides a method of recovering the lignin from biomass. Also, ultra-filtration of the liquid portion of the hydrolysate, provides a means of recovering the hydrolysis enzymes for reuse from the sugar solution which is the precursor for the production of a number of fuels and chemicals.

Further, unlike most water-based pretreatment methods, IL-pretreatment does not produce sugars or their degradation products during incubation step because these are generally hydrolysis products that require presence of water. Sugars are produced only upon enzyme addition. The absence of sugar degradation products that can prove inhibitory to the subsequent processing of the sugars (such as fermentation to alcohol and lactic acid) eliminates the need for the additional step of “conditioning”—in which these inhibitory products are removed—of the hydrolysate.

The time and temperature during the step of IL-incubation of the biomass is optimized to sufficiently swell matrices of the biomass to enhance the penetration of hydrolyzing enzymes and water during the hydrolysis step. The liquid non-solvent for cellulose that is used for washing is water, an alcohol, acetonitrile or a solvent which dissolves the IL and thereby extracts the IL from the biomass. The alcohol may be ethanol, methanol, butanol, or propanol, and wherein the IL is recovered from the liquid non-solvent.

The method may further comprise reusing the recovered IL for treating more biomass.

The method may further comprise recovering the IL from the liquid non-solvent by a method selected from one or more of activated charcoal treatment, distillation, membrane separation, electro-chemical separation techniques, sold-phase extraction and liquid-liquid extraction.

### Recovery of IL/Dehydration of IL

The wash effluent may be collected and the ionic liquid dehydrated by the application of RF energy. The RF energy heats IL faster than it heats water because of a stronger dipole moment in IL. Without being bound to a specific theory, the inventors surprisingly discovered that the ions try to align with the electromagnetic (EM) (e.g., radiofrequency) waves, always changing a dipole moment. The IL heated by RF acts as a substrate for the water to heat and evaporate from the IL wash effluent. Thus, the wash effluent comprising a solvent and ionic liquid may be heated using RF energy. The RF energy drive off the water which may be collected and removed from the wash. The resultant ionic liquid is thus dehydrated (e.g., the water has been removed) and may be reused.

Thus, the ionic liquid is recovered from the liquid non-solvent by application of electromagnetic heating including but not limited to radiofrequency heating, that dehydrates the ionic liquid. The method may further comprise reusing the recovered IL for treating more biomass, optionally wherein at least 90, 91, 92, 93, 94, 95, 96, 97, 98, or 99% of the IL is recovered. After recovery, the ionic liquid may have a water content not exceeding about 1, 2, 3, 4, 5, 6, 7, 8, 9, 10, 11, 12, 13, 14, 15, 16, 17, 18, 19, 20, 21, 22, 23, 24, or 25%.

### Treatment of Biomass Using Combination of Ionic Liquids and Heating

Lignocellulosic biomass may be treated using radio frequency heating in conjunction with ionic liquids (ILs) to facilitate efficient and rapid enzymatic hydrolysis of its carbohydrates. Exemplary ionic liquids (IL) and treatment methods are described in U.S. Pat. No. 8,030,030. Lignocellulosic biomass may be treated utilizing heating by radio frequency, ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof, for effective and amenable conversion of biomass and derived products to renewable fuels, chemicals, and materials. For example, radio frequency heating provides uniform heating and penetration of the biomass.

A method for conversion of the carbohydrates of lignocellulose to sugars with improvements in yield and rate of sugar production using ionic liquid (IL) treatment in combination with RF heating. This treatment strategy substantially improves the efficiency (in terms of yield and reaction rates) of hydrolysis (e.g., saccharification) of lignocellulosic biomass. Other features of this IL-treatment method that have a major impact on the overall economics of sugar production from biomass, in contrast to prior art methods, are its (i) ability to process a variety of lignocellulosic biomass sources with ILs capable of disrupting native cellulose structure (ii) ability to handle large biomass to IL ratios during incubation (iii) ability to accomplish saccharification at very low enzyme loadings (iv) ability to perform well with large biomass particles (v) potential for total recovery (through facile means) and multiple reuse of the IL employed to treat the biomass, (vi) ability to produce a hydrolysate free of compounds that can inhibit the down-stream processing of the constituent sugars, (e.g., ethanol and lactic acid production), and (vii) allows for recovering most of the lignin in biomass following saccharification.

The biomass may be comminuted to smaller sized particles prior to mixing with an ionic liquid and treatment. The biomass may be fed into a chipper, grinder, chopper, shredder, or similar machine to be reduced in size. For example, the biomass may be ground, chopped, or otherwise comminuted to small particles about 0.1-2 mm.

For example, a method for producing sugars from biomass, including but not limited to wheat straw, waste rice straw, kudzu, agricultural waste, municipal waste, corn stover, wood waste, agricultural residues including but not limited to corn stover, wheat straw, bagasse, rice hulls, or rice straw; wood and forest residues including but not limited to pine, poplar, Douglas fir, oak, saw dust, paper/pulp waste, or wood fiber; herbaceous energy crops including but not limited to switchgrass, reed canary grass, or miscanthus; lingocellulosic biomass comprising lignin, cellulose, and hemicellulose; and plant biomass. The biomass may be added to a high solids loading (e.g., >30% w/w). The biomass is mixed with ionic liquid (IL) to swell the biomass but not dissolve it in the IL and heated using radio frequency (RF) energy. Both the mixing with ionic liquid and heating with RF may be monitored for sufficient penetration and uniform heating and the conditions (e.g., time, pressure, heat, intensity of RF energy) may be adjusted as necessary to maintain sufficient penetration and uniform heating of the biomass. Optionally, after the application of RF heating, ultrasonics, electromagnetic heating (EM) (e.g., radiofrequency), convective, conductive heating, or combinations thereof may be used to maintain the temperature of the biomass.

### Electromagnetic (EM) Wave Heating

Biomass products at high solids loadings are relatively poor thermal conductors and most conventional thermal treatment processes rely on heat penetration by conduction from the outside to the inside of the product (surface heating). The processing times can be unacceptably long in industrial scale processing operations. Dielectric heating by microwave or radio-frequency (RF) energy shortens thermal processes because heat is generated by direct interaction between electromagnetic energy and the products. RF-heating, in a similar manner to microwave heating, generates heat volumetrically throughout the product. However, RF treating differs from microwave treatment in that the product is placed between two parallel electrodes and an RF field may be generated in a directional fashion at right angles to the surface of the electrodes.

In addition, the mechanism of dielectric heating with RF field is different from microwave (MW) heating. MW heating occurs mainly via frictional heat generated from the dipolar rotation of free water molecules whereas the predominant mechanism of heating RF is via the depolarization of solvated ions. MW and RF heating also differ in a number of other respects. As frequency and wavelength are inversely proportional, RF (lower frequency) wavelengths (i.e., 11 m at 27.12 MHz in free space) are much longer than MW (higher frequency) wavelengths (i.e., 0.12 m at 2450 MHz in free space). As electrical waves penetrate into materials attenuation occurs, with the result that the energy of the propagating wave decreases exponentially. Penetration depth (dp) is defined as the depth into the material to which the energy is reduced to 1/e (1/2.72) of the surface energy value. This dp is proportional to wavelength. The free-space wavelength in the RF range (e.g., 13.56, 27.12 and 40.68 MHz) is 20-360 times longer than that of commonly used microwave frequencies (e.g., 915 and 2450 MHz), allowing RF energy to penetrate products more deeply than microwave energy. During RF heating, electromagnetic power can penetrate much deeper into samples without surface over heating or hot/cold spots developing which are more likely to occur with MW heating. Thermal processing with RF heating is, therefore, suitable for processing large products/processes. Wang, et al. (2003) Journal of Food Science 68(2): 539-544.

RF heating offers advantages of more uniform heating over the sample geometry due to both deeper level of power penetration and also simpler more uniform field patterns compared to MW heating. In contrast to RF-heating, higher frequency microwaves may provide for greater heating intensity, however, have limits for biomass products when they cannot penetrate deeply enough or provide uniform heating. Power penetration depth decreases with shorter wavelength that is, increasing frequencies. Penetration depths at radio frequencies are of the order of meters and, unless the loss factor is extremely high, through heating may be assured. In the microwave region, on the other hand, the penetration depths become very small, especially when a material is very wet. The wavelength at the RF heating frequencies designated by the Federal Communication Commission (FCC) for industrial heating is 22 to 360 times as great as that of the 2 commonly used microwave frequencies, which allows RF energy to penetrate dielectric materials more deeply than microwaves. Thus, radio frequency heating shows unexpected results in biomass treatment and dielectric materials processing at larger scales and higher levels of solids loading (e.g., about >20% w/w and about >70% w/w).

RF heating has been proven to allow rapid heat transfer throughout dielectric materials as the volumetric heating does not depend on heat transfer through the surface and continues through the boiling point of water and beyond. RF heating is a heating technology that allows for rapid, uniform heating throughout a medium. This technology generates greater energy within the product and throughout its mass simultaneously due to frictional interactions of polar dielectric molecules rotating to an applied external electric field. RF dielectric heating offers several advantages over conventional heating methods in food application, including saving energy by increasing heat efficiency, achieving rapid and even heating, reducing checking, avoiding pollution as there are no byproducts of combustion. Cathcart and Park (1946) first studied the use of RF heating to thaw frozen eggs, fruits, vegetables, and fish. Radio frequency dielectric heating is now widely used in industrial applications such as drying wood logs, textile products (e.g., spools, rovings, skeins), final drying of paper, final dehydration of biscuits at outlets of baking ovens, and melting honey (Barker 1983; Orfeuil 1987).

The problem however with a straight forward use of electromagnetic (EM) (e.g., radiofrequency) wave heating of biomass and ionic liquid complex is the generation of runaway temperatures. In typical uses described above, water absorbs the impinging energy and helps raise the temperature of the complex. As water evaporates, the electromagnetic (EM) (e.g., radiofrequency) waves pass through that part of the material without further energy dissipation. With ionic liquids or complexes containing ions, that do not evaporate or are not meant to evaporate, the setup needs to be much more specifically controlled. The control may include several sensors (e.g., thermocouples, nano-sensors, flow sensors, or other types of sensors) that relay the local conditions so the electromagnetic (EM) (e.g., radiofrequency) unit for that region can be appropriately controlled (e.g., turned on/off or set to a different frequency/power). This setup as such can be used in treatment, hydrolysis (e.g., acid hydrolysis or enzymatic hydrolysis or IL based or a combination thereof) or other reaction environments, whenever the loading of biomass with respect to the other components in the complex is relatively high.

The heating for the treatment of the biomass may comprise two phases: (1) Initial Phase where RF energy is applied to rapidly heat the biomass and (2) Maintenance Phase where of ultrasonics, electromagnetic irradiation (EM) (e.g., radiofrequency), convective, conductive heating, or combinations thereof is applied to maintain the heat of the biomass.

The heating of the biomass by RF may be monitored by a microcontroller and maintained within set parameters of temperature and pressure. For example, the biomass may be maintained at a pre-determined temperature, and additional RF applied when the temperature of the biomass falls below this target temperate and RF is discontinued when the temperature of the biomass exceeds the target temperature. This process may be repeated to maintain an average temperature of the biomass during RF heating.

The inventors surprisingly found that the RF heating may rapidly, uniformly, and effectively heat the biomass allowing for a faster processing time of the biomass. Also, the use of RF heating unexpectedly allowed for higher solids loading (e.g., >30% w/w).

Radio frequency (RF) may comprise a frequency between at least about 3-30 Hz, 30-300 Hz, 300-3000 Hz, 3-30 kHz, 30-300 kHz, 300 kHz-3 MHz, 3-30 MHz, or 30-300 MHz. The radio frequency (RF) may be about 13, 13.56, 27, 27.12, 40, or 40.68 MHz.

The biomass may heated to a temperature of at least about 10° C., 20° C., 30° C., 40° C., 50° C., 60° C., 70° C., 80° C., 90° C., 100° C., 120° C., 130° C., 140° C., 150° C., 200° C., 300° C., 400° C., 60° C.-130° C., 80° C.-175° C., 130° C.-150° C., or 100° C.-240° C.

The radiofrequency may penetrate RF penetrates the biomass to about 0.001 to 2.0 meters thickness. The radiofrequency heating may occur with agitation, either intermittent or continuous.

The biomass may be heated with RF for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds. The biomass may be heated with RF for at least about 1-60 seconds, 1-30 seconds, 1-20 seconds, 5-10 seconds, or 1-10 seconds. The biomass may be heated with RF for at least about 10, 20, 30, 40, 50, 60 seconds. The biomass may be heated with RF for at least about 1, 2, 3, 4, 5, 6, 7, 8, 9, 10, 20, 30, 40, 50, 60, 70, 80, 90, 100, 110, 120, 130, 140, 150, 160, 170, or 180 minutes. The biomass may be heated with RF for at least about 1, 2, 3, 4, 5, 6, 7, 8, 9, 10, 11, 12, 13, 14, 15, 16, 17, 18, 19, 20, 21, 22, 23, or 24 hours. The biomass may be heated with RF for at least about 1, 2, 3, 4, 5, 6, 7, 8, 9, or 10 days. The biomass may be heated with RF for at least about 5-10 seconds, 10-30 seconds, 10-30 minutes, 1-30 minutes, 5-30 minutes, 1-20 minutes, 20 minutes to 2 hours, 5 minutes to 3 hours, 5 minutes to 2 hours, 1-4 hours, 2-4 hours, 1-2 hours, or 3-4 hours.

The biomass may treated at a pressure of at least about 1, 2, 3, 4, 5, 6, 7, 8, 9, 10, 11, 12, 13, 14, 15, 16, 17, 18, 19, 20, 30, 40, 50, 60, 70, 80, 90, or 100 atmospheres (atm).

The ultrasonics used in the methods described herein may be sound waves with high frequency about between 15-40 kHz, 20-30 kHz, 25-35 kHz, or about 15, 20, 30, 35, 35, or 40 kHz) with an amplitude between about amplitude about between 0.0001-0.025 mm. The ultrasonics heating may occur with agitation, either intermittent or continuous.

The biomass may be heated at a power of 100-1,000 W, 1 KW-10 KW, or 5 KW-1 MW.

The biomass may be comminuted to smaller sized particles. The biomass may be comminuted to smaller sized particles prior to mixing with an ionic liquid. The biomass may be comminuted to small particles about 0.1-20 mm, 0.1-2 mm, or about 5 mm in size.

A method for disruption of the structure of a lignocellulosic biomass comprising lignin, cellulose and hemicellulose and treating the disrupted biomass, may comprise incubating the biomass in an ionic liquid (IL) for a sufficient time and temperature to swell the cellulose and hemicellulose without dissolution of the biomass in the IL, optionally heating by radio frequency, ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof; washing the swelled IL-incubated lignocellulosic biomass with a liquid non-solvent for cellulose that is miscible with water and the IL; and treating the incubated and washed lignocellulosic biomass with chemical or biochemical reagents to effect the conversion of the swollen biomass to useful chemicals.

The biomass may heated by radio frequency, ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof to a temperature of at least about 10° C., 20° C., 30° C., 40° C., 50° C., 60° C., 70° C., 80° C., 90° C., 100° C., 120° C., 130° C., 140° C., 150° C., 200° C., 300° C., 400° C., 60° C.-130° C., 80° C.-175° C., 130° C.-150° C., or 100° C.-240° C.

The biomass may be heated by radio frequency, ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof, for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds. The biomass may be heated for at least about 1-60 seconds, 1-30 seconds, 1-20 seconds, 5-10 seconds, or 1-10 seconds. The biomass may be heated for at least about 10, 20, 30, 40, 50, 60 seconds. The biomass may be heated for at least about 1, 2, 3, 4, 5, 6, 7, 8, 9, 10, 20, 30, 40, 50, 60, 70, 80, 90, 100, 110, 120, 130, 140, 150, 160, 170, or 180 minutes. The biomass may be heated with RF for at least about 1, 2, 3, 4, 5, 6, 7, 8, 9, 10, 11, 12, 13, 14, 15, 16, 17, 18, 19, 20, 21, 22, 23, or 24 hours. The biomass may be heated for at least about 1, 2, 3, 4, 5, 6, 7, 8, 9, or 10 days. The biomass may be heated for at least about 5-10 seconds, 10-30 seconds, 10-30 minutes, 1-30 minutes, 5-30 minutes, 1-20 minutes, 20 minutes to 2 hours, 5 minutes to 3 hours, 5 minutes to 2 hours, 1-4 hours, 2-4 hours, 1-2 hours, or 3-4 hours.

The biomass may be processed at a high level of biomass. The biomass may comprise high solids loadings at least about 10%, 20%, 30%, 40%, 50%, 60%, 70%, 80%, 90%, or 95% w/w. The biomass may be loaded at high solids loading at approximately 30% w/w. The inventor surprisingly discovered that the use of electromagnetic heating (e.g., radiofrequency heating, variable frequency electromagnetic heating) allows for the treatment of biomass at high solids loading levels, e.g., >30% w/w.

A system and method for treatment of biomass may employ a variable frequency in the electromagnetic spectrum in combination with an ionic liquid. A system and method for treatment of biomass may employ a variable frequency in the electromagnetic spectrum in combination with an ionic liquid and an acid. The treated biomass may be further processed to yield renewable fuels, chemicals and materials including but not limited to ethanol, butanol, lactic acid, gasoline, biodiesel, methane, hydrogen, electricity, plastics, composites, protein, drugs, fertilizers or other components thereof.

The electromagnetic heating used in the methods and systems described herein may be a variable frequency in the electromagnetic spectrum (e.g., radiofrequency).

### Alkaline Treatment

Following treatment (e.g., incubation with ionic liquid and heating), the treated biomass may be washed to remove the ionic liquid and then treated with alkaline treatment including but not limited to soda, Kraft, sulphite, and/or alkaline process (e.g., NaOH, ammonia, LiOH, Mg(OH)₂, Al(OH)₃, Ca(OH)₂, H₂O₂, NaS, NasCo₃) under extremely mild conditions.

Soda pulping was one of the first chemical pulping methods invented in 1851 by Burgess (USA) and Watts (England). Though kraft or sulfite pulping process took over soda pulping process for generating high strength pulp, soda pulp offers advantages for environmental concerns. As sodium hydroxide is the only used chemical in soda pulping, air pollutants such as hydrogen sulfide (from kraft process) and sulfur dioxide (from sulfite process) are not observed. Process conditions range from treating biomass with 11 to 22% NaOH at 160-200° C., 70 to 130 Psi for 4 to 5 hours of treatment.

Alkaline pretreatments of biomass have been attempted using reagents ammonium, sodium hydroxide and calcium hydroxide starting as early as 1972 to improve the digestibility of cellulose/biomass as efficient feed stock for livestock with improvements of up to 13 to 90%. NaOH, ammonia and Ca(OH)₂ yielded similar digestibility yields based on the studies.

Numerous alkaline pretreatment techniques have been developed for biomass pretreatment over the years. These include sodium hydroxide, sodium carbonate, calcium hydroxide, aqueous ammonia. Alkaline pretreatments delignify biomass and solubilize hemicellulose. The effectiveness of alkaline pretreatment depends on the extent of lignin present. The mechanism of alkaline hydrolysis is believed to be the saponification of intermolecular ester bonds cross-linking xylan hemicelluloses and other components such as lignin. The porosity of the lignocelluosic biomass increases with the removal of the crosslinks thereby increasing the digestibility of cellulose. For example, U.S. Pat. No. 5,693,296 describes the use of several pretreatment conditions.

The present invention provides sequential pretreatments of ionic liquids followed by mild alkaline treatment (e.g., using in either single or combination of NaOH, ammonia, LiOH, MgOH, Ca(OH)₂, H₂O₂, NaS, Na₂CO₃). Ionic liquid pretreatment swells the biomass and whereas mild alkaline treatment conditions separates cellulose pulp from lignin. The decrystallized swelled cellulosic and segregated lignin fractions thus obtained can be rapidly converted to valuable fuels and chemicals in a catalytic process. The inventors surprisingly discovered that the mild alkaline treatment was sufficient to release the lignin from the cellulose pulp and allows greater access to the cellulose pulp by enzymes during the later hydrolysis step. This lead to unexpected improvement in the recovery of enzymes, the reduction of hydrolysis time, and greater yields of sugars.

The alkaline agents may be used at a concentration of about 10-20% by weight. The alkaline agents may be used at a concentration of about 1, 2, 3, 4, 5, 6, 7, 8, 9, 10, 11, 12, 13, 14, 15, 16, 17, 18, 19, or 20% by weight. The alkaline agents may be used at a concentration of about 2, 3, 4, or 5% by weight.

The mild alkaline condition may comprise about a pH 8-13, pH 8-10, pH 9-11, pH 9-10, pH 10-11, pH 9.5-10.5. The pH may be about 8, 8.25, 8.5, 8.75, 9, 9.25, 9.5, 9.75, 10, 10.25, 10.5, 10.75, 11, 11.5, 12, 12.5, or 13.

The alkaline treatment may occur at a temperature of at least about 10° C., 20° C., 30° C., 40° C., 50° C., 60° C., 70° C., 40° C.-60° C., 50° C.-70° C., 50° C.-60° C., or 40° C.-70° C. The alkaline treatment may occur at a temperature of at least about 40° C., 50° C., 70° C., 40° C.-60° C., 50° C.-70° C., 50° C.-60° C., or 40° C.-70° C.

The biomass may undergo alkaline treatment for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes. The biomass may undergo alkaline treatment for at least about 1-60, 1-30, 1-20, 5-10, or 1-10 minutes. The biomass may undergo alkaline treatment for at least about 10, 20, 30, 40, 50, or 60 minutes. The biomass may undergo alkaline treatment for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes. The biomass may undergo alkaline treatment for at least about 5-10, 10-30, 10-30, 1-30, 5-30, 1-20, or 30 minutes.

These conditions are less harsh than the prior methods of soda/kraft pulping conditions which generally last for hours (e.g., 3-5 hours) at high temperature (e.g., 120-150° C.) for weeks (e.g., 3-4 weeks).

### Conversion to Value Added Products

The treated biomass may be washed and then undergo cellulose hydrolysis (cellulolysis) to break down the cellulose and hemicellulose into sugars and free lignin and/or proteins. In the hydrolysis process, the cellulose and hemicellulose may undergo a chemical treatment (e.g., using acids) or a biochemical treatment (e.g., enzymatic digestion). The sugars may then be separated from residual materials (e.g., lignin, proteins). The sugar solution may then be converted to chemicals (e.g., ethanol, lactic acid, succinic acid). Treatment with has a major influence on the reducing the cost in both prior (e.g., size reduction) and subsequent (e.g. enzymatic hydrolysis) operations in sugar production and improving yields.

The following processes may be used to convert biomass (e.g., cellulose, hemicellulose) to value added chemicals (e.g., ethanol). See Corma, et al. (2007) “Chemical Routes for the Transformation of Biomass into Chemicals.” Chem. Rev. 107: 2411-2502. The methods described herein separates the biomass into its main constituents: cellulose, hemicellulose, and lignin. The cellulose and hemicellulose may then be converted (e.g., hydrolysis) to sugars. For example, the hemicellulose may be converted to five and six carbon sugars (e.g., xylose, arabinose) and the cellulose may be converted to six-carbon sugars (e.g., glucose.) The sugars may then be fermented to product products (e.g., ethanol). The proteins and/or lignin may be converted to energy, fuel, plastics, or binders. The cellulose and hemicellulose may undergo a hydrolysis process (cellulolysis), either chemical treatment (e.g., acids) or a biochemical treatment (e.g., enzymatic digestion). Some methods for the chemical processing of cellulose, lignocellulose, and other biomass into chemicals are known in the art. See, e.g., Kobayashi, et al. (2012) Catal. Sci. Technol. 2: 869-883; Ishikawa & Saka (2001) “Chemical Conversion of Cellulose as treated in supercritical methanol.” Cellulose 8(3): 189-195; Tao, et al. (2011) “Catalytic Conversion of cellulose to chemicals in ionic liquid.” Carbohydrate Research 346(1): 58-63; Tao, et al. (2011) Carbohydrate Research 346(1): 58-63; and Binder & Raines (2009) J. Am. Chem. Soc. 131: 1979-1985. These methods may be used in conjunction with the treatment and treatment methods described herein.

### Chemical Conversion to Value Added Products

The chemical treatment may comprise incubation with acids under heat and pressure or a concentrated acid hydrolysis process (e.g., Scholler process). See also Robinson (1995) “A Mild, Chemical Conversion of Cellulose to Hexane and Other Liquid Hydrocarbon Fuels and Additives,” ACS Fuel Chemistry Preprints 40(3): 729 and Binder & Raines (2010) PNAS 107(10): 4516-4521. The cellulose may be treated with alkaline peroxide and then treated with enzymes to break down the cell wall. For example, the biomass may be treated with an ionic liquid to convert the sugars (e.g., glucose and fructose) into 5-hydroxymethylfurfural (HMF). Oxidation of HMF yields 2,5-furandicarboxylic acid.

In other processes, the cellulose and hemicellulose may be converted to 5-hydroxymethylfurfural (HMF) that may be used as a raw material for plastics and fuels. A metal chloride (e.g., chromium chloride) may be used with an ionic liquid to convert the sugars (e.g., glucose and fructose) into HMF. The chemical, a metal chloride known as chromium chloride, converted sugar into highly pure HMF. The metal chlorides and ionic liquid may be reused. Oxidation of HMF yields 2,5-furandicarboxylic acid, which may be used as a replacement for terephthalic acid in the production of polyesters (e.g., polyethylene terephthalate (PET)). See Zhao, et al. (2007) Science 316(5831): 1597-1600.

Further, the cellulose may be degraded by the use of cooperative ionic liquid pairs for combined dissolution and catalytic degradation of cellulose into 2-(diethoxymethyl)furan. See Long, et al. (2011) Green Chem. 13: 2334-2338.

Catalysts may be used in the methods described herein to increase the reaction rate of the reactions. For example, alkali and alkaline earth metal chlorides, and transition metal chlorides (e.g., CrCl₃, FeCl₂, and CuCl₂), and IIIA metal chlorides (e.g., AlCl₃) may be used as catalysts. See, e.g., Peng, et al. (2010) Molecules 15: 5258-5272. Additionally, CoSO₄ may be used as a catalyst in conjunction with an ionic liquid.

Additionally, the sugars produced by the methods described herein may be used to produce succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof.

Also, the treated biomass produced by the methods described herein may be used to produce succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof. Also, the chemical processing of the treated biomass may yield gas productions including but not limited to methane, ethane, CO, CO₂, and H₂.

### Biochemical Conversion to Value Added Products

The biochemical reagent used to convert the washed biomass is an added enzyme. The treating step may comprise adding a buffer comprising enzymes capable of hydrolyzing both cellulose and hemicellulose to the incubated and washed biomass to hydrolyze the cellulose and hemicellulose to sugar. The aqueous buffer may comprise enzymes may comprise an enzyme mixture of cellulases, endo-glucanases, exo-glucanases, and 1-beta-glucosidases. The method may further comprising recovering the enzymes from the hydrolyzed biomass. The enzyme mixture may further may comprise xylanases or arabinases.

In enzymatic hydrolysis, the cellulose is digested into sugar molecules by cellulase enzymes. The lignocellulosic materials may be enzymatically hydrolyzed at mild conditions (e.g., 50° C. and pH 5) to breakdown the cellulose. The methods described herein combine ionic liquid pretreatment with mild alkaline treatment to prepare lignocellulosic biomass for enzymatic hydrolysis to produce monomeric sugars and then chemicals, fuels, and materials. The combination of the ionic liquid pretreatment with mild alkaline treatment has the unexpected result of releasing the lignin from the cellulose and hemicellulose which, in turn, allows for less enzyme to be used for a shorter period of time with greater recovery of the enzyme. Without being bound to a particular theory, the inventors suggest that the release and removal of the lignin from the lignocellulosic biomass allows for greater access of the enzymes to hydrolyze the cellulose and hemicellulose and also eliminates excess lignin which may bind and thus, interfere with, the enzymes.

The enzymes may be used at an amount of 0.5, 1, 2, 3, 4, or 5% enzymes. The enzymes may be used at an amount of 0.5%, 1%, or 2%.

The hydrolysis by enzymes may be for about 1, 2, 3, 4, 5, 6, 7, 8, 9, 10, 11, or 12 hours. The enzymatic hydrolysis may be for about 8-12 hours. This time is greatly reduced from the 24-36 hours or 72-96 hours hydrolysis times of prior hydrolysis methods.

The enzymes may be recovered after hydrolysis. For example, about 60, 70, 80, 90, 91, 92, 93, 94, 95, 96, 97, 98, 99, or 100% of the enzymes may be recovered and, optionally, reused. About 80% or 90% of the enzymes may be recovered and, optionally reused. This is in contrast to prior methods were less than 50% of the enzymes may be recovered. For example, in prior methods after 36 hours of enzyme hydrolysis about 50% of the enzyme was recovered. In contrast, the methods described herein allows for 8-12 hours of enzyme hydrolysis with over 90% recovery of the enzyme. Thus, the methods described herein allow for the run of several “cycles” of enzyme hydrolysis per day thus allowing for an unexpectedly high yield of monomeric sugars from treated biomass. For example, in prior methods over the course of 5 days, 3-4 cycles may have been run with about 50% recovery. In contrast, the methods described herein, over the course of 5 days allows for over 20 cycles with 80-90% recovery of enzymes. This allows for an unexpectedly greater yield of sugars, with less enzymes over the same period of time, resulting in cost savings.

Further, alkali pretreatments at high temperatures and a few hours of processing result in hemi-cellulose and lignin predominantly staying together and cellulose obtained in a separate stream. Alkali pretreatments at milder conditions 50-70° C. needs days to weeks for pretreatments and result in separate lignin stream and cellulose, hemi-cellulose stream. In contrast, the methods described herein comprise mild conditions and short duration processing times with the production of a lignin rich stream and a separate cellulose, hemi-cellulose rich stream. Thus, the methods described herein involve short duration (e.g., minutes) and retain hemicellulose with cellulose stream.

In the enzymatic treatment of the treated biomass, the biomass may be heated to at least about 50-100° C., 55° C., or 70° C.

For example, cellobiohydrolase, exo-1,4-β-glucanase, do-beta-1,4-glucanase, beta-glycosidase, endocellulase, exocellulase, cellobiase, and beta-1,4-glucanase may be used for enzymatic digestion of cellulose. The hemicellulases include but are not limited to laminarinase, lichenase, β-xylosidase, xylanases (e.g., endo-1,4-β-xylanase, xylan 1,4-β-xylosidase, xylan endo-1,3-O-xylosidase, xylan 1,3-β-xylosidase), α-L-arabinofuranosidase, arabianan endo-1,5-α-L-arabinosidase, mannananses (e.g., mannan endo-1,4-β-mannosidase, mannan mannosidase, mannan 1,4-β-mannobisosidase, mannan endo-1,6-β-mannosidase), galactanases, and xylanase may be used for enzymatic digestion of hemicellulase. Jeffries “8. Biodegradation of lignin and hemicelluloses.” Biochemistry of Microbial Degradation pages 233-277. The cellulase, xylanase, and hemicellulase enzymes may be recombinant, including those expressed by recombinant fungi. See Lynd (1996) Annu Rev Energy Environ 21: 403-465.

In the combined hydrolysis and fermentation approach, the cellulose and hemicellulose may be incubated with Clostridium thermocellum which uses its a complex cellulosome to break down cellulose into ethanol, acetate, and lactate.

For ethanol production, the cellulose may undergo cellulolysis processes or gasification. In cellulolysis, the treated lignocellulosic biomass undergoes hydrolysis and then the cellulose may be treated by microbial fermentation. For example, the cellulose may be incubated with Saccharomyces cerevisiae, Zymomonas mobilis, and Escherichia coli, including recombinant microbes, to ferment xylose and arabinose to produce sugars and ethanol. See Jeffries & Jin (2004) Appl Microbiol Biotechnol 63(5): 495-509. The gasification process, a thermochemical approach, the cellulose and hemicellulose is converted into synthesis gas. The carbon monoxide, carbon dioxide and hydrogen may then be incubated with Clostridium ljungdahlii. The Clostridium ljungdahlii ingests carbon monoxide, carbon dioxide, and hydrogen to produce ethanol and water.

Thermostable enzymes may be used in the hydrolysis step. Thermostable enzymes may be stable and active up to about 70° C., as opposed to 55° C. for most commercially available enzymes.

Additionally, the sugars produced by the methods described herein may be used to produce succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof.

Also, the treated biomass produced by the methods described herein may be used to produce succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof. Also, the biochemical processing of the treated biomass may yield gas productions including but not limited to methane, ethane, CO, CO₂, and H₂.

Further, the hemicellulose may be converted to xylose and then to ethanol, xylitol, plastics. The lignin may be converted to fuel, plastics, and binders. The cellulose may be converted to glucose and pulps.

Proceeding now to a description of the drawings, FIG. 2A shows an exemplary series for carrying out steps of a method of the present invention.

One of the following representative ionic liquids 1-n-butyl-3-methylimidazolium chloride (BMIMCl)/1-n-ethyl-3-methyl imidazolium acetate (EMIMAc)/1-ethyl-3-methyl imidazolium propionate (EMIMPr)/1-allyl-3-methyl imidazolium chloride/3-methyl-N-butylpyridinium chloride may be contacted with small particles of biomass 100 (e.g., dry corn stover or poplar (−20+80 mesh sized particles)] for varying times (about 5 minutes to 8 hours) 200. The biomass may be heating using electromagnetic (EM) (e.g., radiofrequency) heating and ultrasonics, electromagnetic (EM) (e.g., radiofrequency), convective, conductive heating, or combinations thereof at about 50° C. to 200° C. as long as the ionic liquid is in molten state during incubation 200. The conditions may be monitored by use of sensors and adjusted to maintain conditions. The biomass may be heated with RF heating at about 27 mHz for at least about 5 seconds to 2 hours. The biomass may then be heated using ultrasonics, electromagnetic (EM) (e.g., radiofrequency), convective, conductive heating, or combinations thereof is for about at least 3-30 minutes or 3-4 hours. The conditions may be monitored and adjusted to maintain uniform heating and sufficient penetration of the biomass by the RF waves.

The treated biomass may then contacted with one of the representative wash-solvents, namely, methanol/ethanol/water/acetonitrile/butanol/propanol 300. The wash-solvent mixes with the IL (in all proportions) and hence is able to extract it from the incubated biomass. The treated biomass may then be separated from the ionic liquid/wash solvent solution by centrifugation. The biomass, stripped off the IL, may then subjected to mild alkaline treatment 400. The IL may be recovered from the wash-solvent and any dissolved biomass components from the wash-step through suitable separation methods including at least one of the following: activated charcoal treatment, distillation, membrane separation, electrochemical separation techniques, solid phase extraction, liquid-liquid extraction, or a combination thereof. The ionic liquid may then be recycled back to the treatment tank. The wash solvent also may be recycled back for reuse in washing IL-incubated biomass. The wash solvent may also be dehydrated by RF heating to dehydrate the wash solvent, driving off the water leaving a dehydrated IL.

The IL may be recovered from the IL/wash solvent mixtures by evaporation of the wash solvent (ethanol and/or water) from the extremely low volatility IL 300. The recovered IL may then be used with no additional cleaning steps in subsequent biomass treatment cycles at constant treatment conditions. The method allows for the repeated reuse of the IL with minimal cleaning which may lead to increased cost savings in IL-treatment.

Residual water in the recycled IL can lower the IL's capacity to sever the inter- and intra-chain hydrogen bonds imparting crystallinity to cellulose. In order to affect swelling of biomass, several of the cellulosic hydrogen-bonds have to be disrupted. Accordingly, it is expect dissolved water to affect IL's performance as a biomass treatment solvent. The admissible water content in IL can affect the economics of the treatment method in two aspects. First, it determines how dry the IL has to be before it can be reused. Second, it determines how dry the biomass has to be during incubation with IL.

In 400, the pretreated biomass is subjected to mild alkaline treatment comprising about 0.5%, 1%, 2%, 3%, 4%, or 5% NaOH or KOH at about a pH of 9-10 for about 30 minutes at 40-50° C. The mild alkaline treatment 400 produces two streams, a lignin rich stream and a cellulose/hemicellulose stream. The lignin rich steam may be directed to further processing 700 to produce chemicals, feedstocks, and fuels. The cellulose/hemicellulose stream rich stream may be directed to hydrolysis 500 for enzymatic hydrolysis to produce constituent monomers and sugars.

After hydrolysis 500, enzymes may be recovered from the hydrolysis reactor and recycled. Due to the less harsh conditions of the mild alkaline treatment, over 80-90% of the enzymes may be recovered. Further, the enzymatic hydrolysis may be for about 8-12 hours. Complete removal of wash solvent (water) is not necessary before the IL is recycled. Many other treatment methods are not amenable to easy recovery of the chemicals employed in the process. Following hydrolysis (saccharification) 500 with an appropriate enzyme mix, capable of converting all the carbohydrates in the pre-treated biomass to sugars. The resultant hydrolysate stream comprising sugars may be directed to further processing for conversion to chemicals. 600

Most of the solids left behind in the saccharification reactor represent the lignin portion of the biomass. This provides a method of recovering the lignin from biomass 700 Also, ultra-filtration of the liquid portion of the hydrolysate, provides a means of recovering the hydrolysis enzymes for reuse from the sugar solution which is the precursor for the production of a number of fuels and feedstock 700.

Steps 200, 300, and/or 400 may be repeated. Further, steps 200 and/or 400 may be carried out in batch or continuous form. The goal of treatment 200 is not achieving any dissolution of lignocellulose, but treatment of the pretreated biomass for sufficient time to release lignin and swell the remaining biomass structure to enhance the hydrolysis rate and conversion of cellulose and hemicellulose to their constituent sugars 500.

FIG. 2B shows an exemplary series for carrying out steps of a method of the present invention.

One of the following representative ionic liquids 1-n-butyl-3-methylimidazolium chloride (BMIMCl)/1-n-ethyl-3-methyl imidazolium acetate (EMIMAc)/1-ethyl-3-methyl imidazolium propionate (EMIMPr)/1-allyl-3-methyl imidazolium chloride/3-methyl-N-butylpyridinium chloride may be contacted with small particles of biomass 101 (e.g., dry corn stover or poplar (−20+80 mesh sized particles)] for varying times (about 5 minutes to 8 hours) to form a biomass 201. Heating of the biomass may be carried out by first electromagnetic (EM) (e.g., radiofrequency) heating to reach a target temperature or temperature range (e.g., 50° C.-220° C.) 201 and then heating using ultrasonics, electromagnetic (EM) (e.g., radiofrequency), convective, conductive heating, or combinations thereof at about 50° C. to 200° C. for 3-30 minutes or 3-4 hours 201.

The treated biomass may then contacted with one of the representative wash-solvents, namely, methanol/ethanol/water/acetonitrile/butanol/propanol 301. The wash-solvent mixes with the IL (in all proportions) and hence is able to extract it from the incubated biomass. The treated biomass may then be separated from the ionic liquid/wash solvent solution by centrifugation. The biomass, stripped off the IL, may then hydrolyzed with a cellulase system 501.

The IL may be recovered from the wash-solvent and any dissolved biomass components from the wash-step through suitable separation methods including at least one of the following: activated charcoal treatment, distillation, membrane separation, electrochemical separation techniques, solid phase extraction, liquid-liquid extraction, or a combination thereof. The ionic liquid may then be recycled back to the treatment tank. The wash solvent also may be recycled back for reuse in washing IL-incubated biomass. The IL may be recovered from the IL/wash solvent mixtures by evaporation of the wash solvent (ethanol and/or water) from the extremely low volatility IL 301. The wash solvent may also be dehydrated by RF heating to dehydrate the wash solvent, driving off the water leaving a dehydrated IL 800. The recovered IL may then be used with no additional cleaning steps in subsequent biomass treatment cycles at constant treatment conditions. The method allows for the repeated reuse of the IL with minimal cleaning which may lead to increased cost savings in IL-treatment.

In 401, the pretreated biomass is subjected to mild alkaline treatment comprising about 0.5%, 1%, 2%, 3%, 4%, or 5% NaOH or KOH at about a pH of 9-10 for about 30 minutes at 40-50° C. The mild alkaline treatment 401 produces two streams, a lignin rich stream and a cellulose/hemicellulose stream.

The lignin rich steam may be directed to further processing 701 to produce chemicals, feedstocks, and fuels.

The cellulose/hemicellulose stream rich stream may be directed to hydrolysis 501 for enzymatic hydrolysis to produce constituent monomers and sugars.

After hydrolysis 501, enzymes may be recovered from the hydrolysis reactor and recycled. Complete removal of wash solvent (water) is not necessary before the IL is recycled. Many other treatment methods are not amenable to easy recovery of the chemicals employed in the process. Following hydrolysis (saccharification) 501 with an appropriate enzyme mix, capable of converting all the carbohydrates in the pre-treated biomass to sugars. The resultant hydrolysate stream comprising sugars may be directed to further processing for conversion to chemicals 601.

Most of the solids left behind in the saccharification reactor represent the lignin portion of the biomass. This provides a method of recovering the lignin from biomass 701. Also, ultra-filtration of the liquid portion of the hydrolysate, provides a means of recovering the hydrolysis enzymes for reuse from the sugar solution which is the precursor for the production of a number of fuels and feedstock 701.

The conditions may be monitored by use of sensors and adjusted to maintain conditions 201. The conditions may be monitored and adjusted to maintain uniform heating and sufficient penetration of the biomass by the RF waves. Steps 201, 301, and/or 401 may be repeated. Further, steps 201, 301, and/or 401 may be carried out in batch or continuous form.

After hydrolysis, the enzymes may be reclaimed. The inventor surprisingly discovered that the use of mild alkaline treatment conditions allows for the recovery of a high percentage of enzymes (e.g., >90%) and their reuse in more cycles than prior methods (e.g., 16-20 cycles versus 1 cycle of hydrolysis). The inventor surprisingly discovered that ionic liquid pretreatment disrupts the crystallinity of cellulose rapidly for effective hydrolysis. However, the inventors found that the ionic liquid pretreatment results in the swelling of the biomass and does not remove the lignin cladding, though disrupting it. See FIG. 1A. Therefore, this remaining lignin can act as a physical barrier for enzymatic hydrolysis of cellulosic and hemicellulosic sugars as it sheaths and covers it, slowing enzymatic hydrolysis (e.g., preventing the enzymes from reaching the cellulose and hemicellulose). Lignin also binds to the enzymes reducing their activity and further reducing the ability to recover and reuse the enzymes. Therefore, removal of lignin from swollen biomass can enhance hydrolysis rates by providing more direct access to cellulosic chains and severely reducing the scope of enzyme loss due to lignin binding.

The current method of combining ionic liquid pretreatment with mild alkaline processing benefits from either alone, or the usually more harsh alkaline treatments used in the art. For example, optimal lime pretreatment conditions were ascertained to be 4 weeks aeration of biomass with lime (1 gram of lime per gram of raw biomass at 55° C. in 91% glucan and 51% xylan yields. See “Lime Pretreatment and Enzymatic Hydrolysis of Corn Stover.” A Dissertation by Se Hoon Kim (2004) Texas A&M University. This is in contrast with over 90% glucan yield and over 60% xylan yield after ionic liquid pretreatment plus mild alkaline treatment at 75° C. for 60 minutes. Thus, the methods described herein reaches the same yields as a conventional lime pretreatment in a matter of minutes rather than weeks.

Further, thermophilic enzymes may be used in the hydrolysis step (e.g., enzymes stable and active at about 70° C.). The use of thermophilic enzymes allows for the hydrolysis step to be run at a higher temperature and improves efficiency and yield of the hydrolysis step. For example, mixtures of thermophilic endo- and exo-glycoside hydrolases may be active at high temperatures and acidic pH. The thermophilic enzymes may be isolated from thermophilic bacteria including but not limited to Sulfolobus solfataricus, Alicyclobacillus acidocaldarius, and Thermus thermophilus. Also, thermophilic cellulases may be used.

Although certain manufacturers, model names and numbers are given for machinery used in the invention, other machinery may be substituted, as would be appreciated by those skilled in the art.

Although certain ranges are provided for the temperature, conveyor speed, electromagnetic (EM) (e.g., radiofrequency) wave intensity, and pressure characteristics, these can be varied based on the particular volumes desired, space requirements and other needs. After reading this specification, one skilled in the art will understand that the selection of working or optimum numbers for these variables may be made once the plant and overall process parameters of a particular processing installation are known.

Additionally, although preferred systems are disclosed for controlling the temperature of the biomass, these may be varied. These may be varied by substituting, depending on normal plant considerations of energy cost, plant lay-out and the like, and generally the temperature values used in the process tolerate some ongoing variability due to, for instance, changes in ambient plant temperatures and other related factors.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

Although methods and materials similar or equivalent to those described herein may be used in the invention or testing of the present invention, suitable methods and materials are described herein. The materials, methods and examples are illustrative only, and are not intended to be limiting.

The invention now being generally described, it will be more readily understood by reference to the following examples, which are included merely for purposes of illustration of certain aspects and embodiments of the present invention, and are not intended to limit the invention.

### EXAMPLES

### Example 1

### Treatment of Wheatstraw

Wheatstraw was comminuted and then subjected to 25% ionic liquid pretreatment for about 2.15 hours. The pretreated wheatstraw was then divided in two groups, the first group then underwent no further treatment before about 2.5 hours of enzymatic hydrolysis and the second group underwent mild alkaline treatment at 5% sodium hydroxide for about 1 hour at about 75° C. The results are shown in Table 1.

TABLE 1
Wheatstraw IL pretreatment with and without mild alkaline treatment.

| | | | | Hydrolysis Time | | Hydrolysis Time | |
|---|---|---|---|---|---|---|---|
| | Ionic liquid | | | (hours) | | (hours) | |
| | pretreatment | Caustic | % | 6 | | 13 | |
| Biomass | Condition | Conditions | Enzymes | % Glucan | % Xylan | % Glucan | % Xylan |
| Wheatstraw | 25% PT WS | None | 2.5 | 44 | 26 | 49 | 30 |
| | 2.15 hours | | | | | | |
| Wheatstraw | 25% PT WS | 75 C. 1 hour, | 2.5 | 81 | 36 | 84 | 38 |
| | 2.15 hours | 5% C. Treat | | | | | |

The combination of the ionic liquid pretreatment and mild alkaline treatment surprisingly improved the yield of glucan and xylan from wheatstraw (lignocellulosic biomass) in a shorter period of time. Thus, the combination of the ionic liquid pretreatment and mild alkaline treatment may be used to improve the enzymatic hydrolysis of the treated biomass in a shorter period of time than prior art methods.

### Example 2

### Comparison of Wheatstraw Treatment with and without Pretreatment and/or Alkaline Treatment

Wheatstraw was comminuted and then divided into two groups. One group of wheatstraw was subjected to 25% ionic liquid pretreatment for about 2.15 hours and the other received no pretreatment. The pretreated and not-pretreated wheatstraw was then divided in two groups, the first group then underwent mild alkaline treatment with sodium hydroxide for about 1 hour at about 75° C. for about 15 minutes at about 50° C. All of the groups underwent either x enzyme or 2× enzyme hydrolysis. The results are shown in Table 2.

TABLE 2
Wheatstraw Processing with and without IL pretreatment and/or mild alkaline
treatment.

| | | | | | Hydrolysis Time | | Hydrolysis Time | | Hydrolysis Time | |
|---|---|---|---|---|---|---|---|---|---|---|
| | | | | | (hours) | | (hours) | | (hours) | |
| | Ionic | | | | 6 | | 13 | | 24 | |
| | liquid | | | % | % | % | % | % | % | % |
| Biomass | PT? | Conditioning | Enzymes | Hydrolysis | Glucan | Xylan | Glucan | Xylan | Glucan | Xylan |
| Wheatstraw | Yes | 75 C. 1 hr, C. | x | 5.5 | 70 | 48 | 90 | 62 | 96 | 67 |
| Wheatstraw | Yes | 75 C. 1 hr, C. | x | 5.5 | 75 | 51 | 95 | 65 | | |
| Wheatstraw | Yes | 50 C., 15 min | 2x | 5.5 | 84 | 69 | 98 | 79 | 103 | 86 |
| Wheatstraw | Yes | None | x | 5.5 | 53 | 50 | 69 | 60 | 75 | 60 |
| Wheatstraw | No | 75 C. 1 hr, C. | x | 5.5 | 15 | 7 | 19 | 9 | 22 | 11 |
| Wheatstraw | No | None | x | 5.5 | 6 | 3 | 9 | 4 | 10 | 6 |

The combination of the ionic liquid pretreatment and mild alkaline treatment surprisingly improved the yield of glucan and xylan from wheatstraw (lignocellulosic biomass) in a shorter period of time. As seen in Table 2, the lack of either IL pretreatment or mild alkaline treatment reduces the yield of sugars (e.g., glucan, xylan) from hydrolysis. Thus, the combination of the ionic liquid pretreatment and mild alkaline treatment may be used to improve the enzymatic hydrolysis of the treated biomass in a shorter period of time than prior art methods.

### Example 3

### Comparison of Wheatstraw Treatment with and without Pretreatment and/or Alkaline Treatment

Enzymatic hydrolysis results of wheat straw biomass after 13 hours of hydrolysis at 2× Enzyme dosage. Four conditions were compared: (1) Ionic liquid pretreatment of wheatstraw with mild alkaline processing at 75° C. 1 hour, (2) Ionic liquid pretreatment of wheatstraw alone, (3) mild alkaline processing at 75° C. 1 hour with no ionic liquid pretreatment and (4) untreated wheat straw. These four conditions were compared for the percentage of glycan and xylan conversion to monomeric sugars. See FIG. 4. Thus, either the ionic liquid pretreatment or mild alkaline treatment may improve the yield of monomeric sugars. However, the combination of both the ionic liquid pretreatment and mild alkaline treatment showed a greater than additive effect in the yield of monomeric sugars from hydrolysis. Further, the pretreatment and mild alkaline treatment processing time is measured in a matter of minutes to hours instead of days to weeks with prior methods.

### Example 4

### Comparison of Wheatstraw Treatment with and without Pretreatment and/or Alkaline Treatment

Wheatstraw was comminuted and then divided into different groups that compared ionic liquid pretreatment in combination with various alkaline agents for the yield from enzymatic hydrolysis. All of the groups underwent enzyme hydrolysis and the amount of glucan and xylan after 12 hours and 24 hours was examined. The results are shown in Table 3.

TABLE 3

| | | | | | % Alkali | | Enzymatic Hydrolysis | | | | |
|---|---|---|---|---|---|---|---|---|---|---|---|
| | | | | Caustic | (w/w) | | | 12 hrs | | 24 hrs | |
| | | Ionic Liquid | | Conditions- | (w.r.t. Dry | Enzyme | Temp | % | % | % | % |
| Sample | Biomass | Pretreatment | Caustic | time, temp | Biomass) | Loading | (° C.) | Glucan | Xylan | Glucan | Xylan |
| 1 | Wheat Straw | Yes | None | | 0.00% | 0 | 0 | 61% | 51% | 71% | 59% |
| 2 | Wheat Straw | Yes | NaOH | 60 min, 50 C. | 16.65% | 2% | 50 | 96% | 54% | 99% | 62% |
| 3 | Wheat Straw | Yes | NaOH | 60 min, 50 C. | 2.20% | 2% | 50 | 73% | 62% | 80% | 68% |
| 4 | Wheat Straw | Yes | NaOH | 60 min, 50 C. | 8.75% | 2% | 50 | 81% | 60% | 89% | 67% |
| 4 | Wheat Straw | No | NaOH | 60 min, 50 C. | 8.75% | 2% | 50 | 33% | 35% | 40% | 38% |
| 5 | Wheat Straw | Yes | KOH | 60 min, 50 C. | 8.75% | 2% | 50 | 84% | 69% | 92% | 77% |
| 5 | Wheat Straw | No | KOH | 60 min, 50 C. | 8.75% | 2% | 50 | 27% | 21% | 33% | 23% |
| 6 | Wheat Straw | Yes | Ca(OH)₂ | 60 min, 50 C. | 8.75% | 2% | 50 | 91% | 78% | 98% | 91% |
| 6 | Wheat Straw | No | Ca(OH)₂ | 60 min, 50 C. | 8.75% | 2% | 50 | 31% | 27% | 36% | 31% |
| 7 | Wheat Straw | Yes | Mg(OH)₂ | 60 min, 50 C. | 8.75% | 2% | 50 | 62% | 53% | 66% | 58% |
| 8 | Wheat Straw | Yes | Al(OH)₃ | 60 min, 50 C. | 8.75% | 2% | 50 | 68% | 56% | 74% | 61% |

The combination of the ionic liquid pretreatment and mild alkaline treatment surprisingly improved the yield of glucan and xylan from wheatstraw (lignocellulosic biomass) in a shorter period of time. As seen in Table 3, the lack of either IL pretreatment or mild alkaline treatment reduces the yield of sugars (e.g., glucan, xylan) from hydrolysis. Thus, the combination of the ionic liquid pretreatment and mild alkaline treatment may be used to improve the enzymatic hydrolysis of the treated biomass in a shorter period of time than prior art methods.

Those skilled in the art will recognize, or be able to ascertain using no more than routine experimentation, many equivalents to the specific embodiments of the invention described herein. Such equivalents are intended to be encompassed by the following claims.

1. A method for the treatment of lignocellulosic biomass comprising
(a) mixing lignocellulosic biomass with an ionic liquid for a sufficient time and temperature to swell the lignocellulosic biomass without dissolution of the lignocellulosic biomass in the ionic liquid; and
(b) treating the swelled lignocellulosic biomass under mild alkaline treatment for about 1-60 minutes to separate the lignin from the cellulose and hemicellulose.

2. The method of claim 1, wherein said biomass is washed after step (a) and before step (b).

3. The method of claim 1, wherein said lignocellulosic biomass is agricultural residue, wood and forest residue, kudzu, herbaceous energy crop, lingocellulosic biomass comprising lignin, cellulose, and hemicellulose, plant biomass, or mixtures thereof.

4. The method of claim 1, wherein said mild alkaline treatment comprises the addition of an alkaline agent.

5. The method of claim 4, wherein said alkaline agent is NaOH, aqueous ammonia, LiOH, Mg(OH)₂, Al(OH)₃, Ca(OH)₂, H₂O₂, NaS, Na₂CO₃, or a combination thereof.

6. The method of claim 4, wherein said alkaline agent is added at a concentration of 10-20% by weight.

7. The method of claim 4, wherein said mild alkaline condition comprise a pH of about 8-11.

8. The method of claim 4, wherein said alkaline treatment is at a temperature of at least about 40° C.-70° C.

9. (canceled)

10. The method of claim 1, wherein the biomass in step (a) or step (b) is subjected to additional heating with agitation, ultrasonic heating, electromagnetic (EM) heating, convective heating, conductive heating, microwave irradiation, or a combination thereof.

11. The method of claim 10, wherein said additional heating comprises intermittent agitation during heating.

12. The method of claim 1, wherein the ionic liquid is molten at a temperature ranging from about 10° C. to 160° C. and comprises cations or anions.

13. The method of claim 12, wherein the ionic liquid comprises a cation structure that includes ammonium, sulfonium, phosphonium, lithium, imidazolium, pyridinium, picolinium, pyrrolidinium, thiazolium, triazolium, oxazolium, or combinations thereof.

14. The method of claim 13, wherein the ionic liquid comprises a cation selected from imidazolium, pyrrolidinium, pyridinium, phosphonium, ammonium, or a combination thereof.

15. The method of claim 14, wherein the ionic liquid (IL) is 1-n-butyl-3-methylimidazolium chloride, 1-allyl-3-methyl imidazolium chloride, 3-methyl-N-butylpyridinium chloride, 1-ethyl-3-methyl imidazolium acetate, 1-ethyl-3-methyl imidazolium propionatem, or combinations thereof.

16. The method of claim 1, wherein said hydrolyzing the cellulose and hemicellulose comprises treating said cellulose and hemicellulose with a chemical or biochemical reagent to convert the cellulose and hemicellulose to sugars.

17. The method of claim 16, wherein said sugars are hexose and/or pentose sugars.

18. The method of claim 16, wherein said biochemical reagent is an enzyme.

19. The method of claim 18, wherein said enzyme is recovered.

20. The method of claim 19, wherein at least 90, 91, 92, 93, 94, 95, 96, 97, 98, or 99% of the enzyme is recovered.

21. The method of claim 18, wherein said enzyme is reused.

22. The method of claim 21, wherein said enzyme is reused for about 16-20 hydrolysis cycles.

23. The method of claim 16, wherein said sugars are converted to renewable fuels, chemicals, and materials.

24. (canceled)

25. A method for conversion of the carbohydrates of lignocellulosic biomass to sugars comprising
(a) mixing lignocellulosic biomass in an ionic liquid (IL) swell but not dissolve the biomass;
(b) applying radio frequency (RF) heating to the lignocellulosic biomass to heat to a target temperature range;
(c) applying ultrasonics, electromagnetic (EM), convective, conductive heating, or combinations thereof, to the lignocellulosic biomass to maintain the lignocellulosic biomass at said target temperature range of about 50-220° C.;
(d) washing the treated lignocellulosic biomass;
(e) subjecting said lignocellulosic biomass to mild alkaline treatment for about 1-60 minutes to release lignin from the cellulosic components;
(f) washing the treated lignocellulosic biomass;
(g) recovering said lignin; and
(h) hydrolyzing the cellulosic components to yield sugars.

26. A method for treatment of lignocellulosic biomass comprising
(a) incubating a biomass in a sufficient amount of an ionic liquid (IL) for a sufficient time and temperature to swell the lignocellulosic biomass without dissolution of the lignocellulosic biomass in the IL;
(b) applying radio frequency (RF) heating to the lignocellulosic biomass to heat to a target temperature range;
(c) applying ultrasonic heating to the lignocellulosic biomass to maintain the biomass at said target temperature range;
(d) washing the pretreated lignocellulosic biomass;
(e) subjecting said lignocellulosic biomass to mild alkaline treatment for about 1-60 minutes to release lignin from the cellulosic components;
(f) washing the treated lignocellulosic biomass with a liquid non-solvent for cellulose that is miscible with water and the IL;
(g) recovering said lignin; and
(h) contacting said washed treated lignocellulosic biomass with an aqueous buffer comprising enzymes capable of hydrolyzing cellulose and hemicellulose to produce sugars.

27. The method of claim 1, wherein said method further comprises recovering the lignin.

28. The method of claim 1, wherein said method further comprises hydrolyzing the cellulose and hemicellulose to produce sugars.
